(19) United States
(12) Patent Application Publication
(10) Pub. No.: US 2023/0365918 A1
(43) Pub. Date: Nov. 16, 2023

(54) VINEGAR AND METHOD FOR MANUFACTURING THE SAME

(71) Applicant: MIZKAN HOLDINGS CO., LTD., Aichi (JP)

(72) Inventors: Atsushi Ishikawa, Handa-shi (JP); Masachika Takaoka, Handa-shi (JP)

(73) Assignee: MIZKAN HOLDINGS CO., LTD., Aichi (JP)

(21) Appl. No.: 18/360,791

(22) Filed: Jul. 27, 2023

Related U.S. Application Data
(63) Continuation-in-part of application No. PCT/JP2022/002686, filed on Jan. 25, 2022.

(30) Foreign Application Priority Data
Jan. 29, 2021 (JP) 2021-013905

(51) Int. Cl. C12N 1/20; C12J 1/00

(52) U.S. Cl. CPC C12N 1/20; C12J 1/00

(57) ABSTRACT

A method for manufacturing an acetic acid bacterium culture and the acid bacterium culture, the acid bacterium culture contains PS1 (Glc:Gal:Man:GlcA=6.0:5.0 to 6.5:0.05 to 0.4:0.05 to 0.4) and/or PS2 (Glc:Rha:Man:GlcA=4.0:1.5 to 2.5:0.05 to 0.4:0.05 to 0.4) which are/is water-soluble polysaccharide(s). The method includes a fermentation step S₁: a step of culturing acetic acid bacteria belonging to the genus Kozakia to give a first culture containing PS1, or culturing acetic acid bacteria belonging to the genus Komagataeibacter to give a first culture containing PS2, or culturing acetic acid bacteria belonging to the genus Kozakia and acetic acid bacteria belonging to the genus Komagataeibacter to give a first culture containing PS1 and PS2.

### CROSS-REFERENCE TO RELATED APPLICATIONS

The present application is a continuation-in-part which claims priority to International Application No. PCT/JP2022/002686, filed Jan. 25, 2022, the disclosure of which is hereby expressly incorporated by reference in its entirety.

### BACKGROUND OF THE INVENTION

### (1) Field of the Invention

The present invention relates to a vinegar and a method for manufacturing the same. More particularly, the present invention relates to a vinegar having a high viscosity and a method for manufacturing the same.

### (2) Description of Related Art

Conventionally, polysaccharides produced by certain acetic acid bacteria are known to impart viscous properties to foods. JP S62-044164 A discloses a method for obtaining a viscous vinegar by a fermentation step of producing a viscous substance and acetic acid fermentation of the obtained culture.

### SUMMARY OF THE INVENTION

On the other hand, for example, liquid seasonings having viscous properties such as dipping sauce, sauce, dressing, and ketchup are known as foods having a sour taste and viscosity. These viscous liquid seasonings are generally required to have a balance between a sour taste and viscosity. Therefore, in the preparation of these viscous liquid seasonings, vinegar can be used to impart a sour taste thereof. However, vinegar is generally a food having a low viscosity, and can increase a sour taste, but is difficult to contribute to an increase in viscosity, and tends to be a factor that decreases viscosity in a viscous liquid seasoning.

On the other hand, it is also possible to increase viscosity by adding a separate thickener or the like, but, actually, there is an increasing number of cases where it is required to manufacture foods without using additives, in recent years. In addition, it is technically difficult to thicken vinegar by post-addition as described in JP S62-044164 A, and it is difficult to thicken vinegar afterwards.

In this respect, it is possible to use the technique disclosed in JP S62-044164 A in response to the above request. That is, it is conceivable to use a viscous vinegar obtained by the method disclosed in JP S62-044164 A. However, as shown in the Examples of JP S62-044164 A, the vinegar obtained by this technique has a viscosity of about 700 mPa·s, and, actually, no vinegar having a higher viscosity than this viscosity has been produced.

The present invention has been made in view of the above circumstances, and an object thereof is to provide a vinegar having a viscosity higher than that of a conventional vinegar and a method for manufacturing the same.

Specifically, the present invention is as follows.

[1] A vinegar containing PS1 below and/or PS2 below which are/is water-soluble polysaccharide(s):
PS1: a water-soluble polysaccharide in which constituent sugars are glucose, galactose, mannose, and glucuronic acid, and a constituent ratio among the constituent sugars is 6.0:5.0 to 6.5:0.05 to 0.4:0.05 to 0.4
PS2: a water-soluble polysaccharide in which constituent sugars are glucose, rhamnose, mannose, and glucuronic acid, and a constituent ratio among the constituent sugars is 4.0:1.5 to 2.5:0.05 to 0.4:0.05 to 0.4.

[2] The vinegar according to [1], which has a viscosity of 1000 mPa·s or more and an acidity of 0.5% or more.

[3] The vinegar according to [1] or [2], wherein a concentration of the PS1 is 5.2 g/L or more, or a concentration of the PS2 is 3.5 g/L or more.

[4] A method for manufacturing a vinegar, including two fermentation steps, S₁ below and S₂ below:
S₁: a fermentation step of culturing acetic acid bacteria selected from acetic acid bacteria belonging to the genus Acetobacter, acetic acid bacteria belonging to the genus Gluconobacter, acetic acid bacteria belonging to the genus Kozakia, acetic acid bacteria belonging to the genus Komagataeibacter, and acetic acid bacteria belonging to the genus Gluconacetobacter in a first medium to give a first culture containing a water-soluble polysaccharide selected from PS1 below and PS2 below
S₂: a fermentation step of culturing acetic acid bacteria selected from acetic acid bacteria belonging to the genus Acetobacter, acetic acid bacteria belonging to the genus Gluconobacter, acetic acid bacteria belonging to the genus Kozakia, acetic acid bacteria belonging to the genus Komagataeibacter, and acetic acid bacteria belonging to the genus Gluconacetobacter in a second medium containing the first culture to give a second culture containing acetic acid and the water-soluble polysaccharide.
PS1: a water-soluble polysaccharide in which constituent sugars are glucose, galactose, mannose, and glucuronic acid, and a constituent ratio among the constituent sugars is 6.0:5.0 to 6.5:0.05 to 0.4:0.05 to 0.4
PS2: a water-soluble polysaccharide in which constituent sugars are glucose, rhamnose, mannose, and glucuronic acid, and a constituent ratio among the constituent sugars is 4.0:1.5 to 2.5:0.05 to 0.4:0.05 to 0.4.

[5] The method for manufacturing a vinegar according to [4], wherein the acetic acid bacteria used in the fermentation step S₁ and the acetic acid bacteria used in the fermentation step S₂ are common with each other.

[6] The method for manufacturing a vinegar according to [4] or [5], wherein the second culture has a viscosity of 1000 mPa·s or more and an acidity of 4% or more.

[7] The method for manufacturing a vinegar according to any one of [4] to [6], wherein the first medium contains fructose.

[8] The method for manufacturing a vinegar according to any one of [4] to [7], wherein the first medium contains at least fructose of fructose and glucose, and
wherein a proportion R_{FRU} of fructose is more than 50 mass % and 100 mass % or less when a total proportion of fructose and glucose is 100 mass %.

The vinegar of the present invention itself has a high viscosity, and can therefore exhibit excellent adhesiveness to foods when used as a viscous liquid seasoning. It can also be used as a raw material for a viscous liquid seasoning having a sour taste. That is, it can be used as a food raw material capable of imparting a sour taste and a vinegar flavor while suppressing a decrease in viscosity.

According to the method for manufacturing a vinegar of the present invention, a vinegar having a viscosity of 1000 mPa·s or more and an acidity of 4 or more can be manufactured. In addition, a vinegar having a viscosity of 1000 mPa·s or more and an acidity of 4 or more can be efficiently manufactured.

### DETAILED DESCRIPTION OF PREFERRED EMBODIMENTS

### [1] Vinegar

The vinegar of the present invention is characterized by containing PS1 and/or PS2 which are/is water-soluble polysaccharide(s).

The “PS1” is a polysaccharide having a constituent sugar ratio glucose:galactose:mannose:glucuronic acid=6.0:5.0 to 6.5:0.05 to 0.4:0.05 to 0.4, and is a heteropolysaccharide. That is, constituent sugars are glucose, galactose, mannose, and glucuronic acid, and a constituent ratio among the constituent sugars is 6.0:5.0 to 6.5:0.05 to 0.4:0.05 to 0.4. Also, PS1 is a water-soluble polysaccharide contained in the present vinegar in a state of being dissolved in water because of its water solubility. Further, PS1 exhibits acidity and is an acidic polysaccharide.

PS1 can be contained in the vinegar by a method which will be described later. That is, PS1 can be contained in the vinegar through two steps, i.e., a fermentation step S₁ and a fermentation step S₂. This method will be described later.

In a case where PS1 is contained in the vinegar, a concentration thereof is not limited, but is preferably 5.2 g/L or more. The concentration is more preferably 6.3 g/L or more, still more preferably 7.0 g/L or more, still more preferably 9.0 g/L or more, and particularly preferably 12.0 g/L or more. Due to PS1 being contained at a concentration of 5.2 g/L or more, an acidity of 0.5% or more and a viscosity of 1000 mPa·s or more can be achieved.

On the other hand, an upper limit value of the concentration is not limited, and the concentration is more preferably higher, but is usually 50 g/L or less, and may be 45 g/L or less, 35 g/L or less, 25 g/L or less, or 20 g/L or less.

The concentration of PS1 is measured by a method shown in the Examples which will be described below.

The “PS2” is a polysaccharide having a constituent sugar ratio glucose:rhamnose:mannose:glucuronic acid=4.0:1.5 to 2.5:0.05 to 0.4:0.05 to 0.4, and is a heteropolysaccharide. Also, PS2 is a water-soluble polysaccharide contained in the present vinegar in a state of being dissolved in water because of its water solubility. Further, PS2 exhibits acidity and is an acidic polysaccharide.

PS2 can be contained in the vinegar by a method which will be described later. That is, PS1 can be contained in the vinegar through two steps, i.e., a fermentation step S₁ and a fermentation step S₂. This method will be described later.

In a case where PS2 is contained in the vinegar, a concentration thereof is not limited, but is preferably 3.5 g/L or more. The concentration is more preferably 5.5 g/L or more, still more preferably 6.0 g/L or more, still more preferably 8.0 g/L or more, and particularly preferably 10.0 g/L or more. Due to PS2 being contained at a concentration of 3.5 g/L or more, an acidity of 0.5% or more and a viscosity of 1000 mPa·s or more can be achieved.

On the other hand, an upper limit value of the concentration is not limited, and the concentration is more preferably higher, but is usually 30 g/L or less, and may be 25 g/L or less, 20 g/L or less, 15 g/L or less, or 10 g/L or less.

The concentration of PS2 is measured by a method shown in the Examples which will be described below.

The viscosity of the vinegar of the present invention can be 1000 mPa·s or more. That is, the vinegar having a viscosity of 1000 mPa·s or more itself has a high viscosity, and can therefore exhibit excellent adhesiveness to target foods when used as a viscous liquid seasoning. It can also be used as a raw material for a viscous liquid seasoning having a sour taste. That is, it can be used as a raw material for imparting a sour taste or a vinegar flavor, in the manufacture of a viscous liquid seasoning (for example, having a viscosity of 1000 to 30000 mPa·s, further 1500 to 20000 mPa·s, further 2000 to 10000 mPa·s, and further 2500 to 5000 mPa·s) such as sauce, ketchup, dipping sauce, or dressing. In particular, when used as such a raw material, it can impart a sour taste and a vinegar flavor while suppressing a decrease in viscosity of the viscous liquid seasoning.

The viscosity of the vinegar is sufficiently 1000 mPa·s or more, but can be further 1500 mPa·s or more, further 2000 mPa·s or more, and further 2500 mPa·s or more (usually, 4500 mPa·s or less).

The viscosity of the vinegar is a viscosity measured using a B-type viscometer under conditions of 25° C., 30 rpm, and rotor No. 3.

The acidity of the vinegar of the present invention can be 0.5% (w/v) or more. Further, the acidity is preferably 1% or more, more preferably 2% or more, still more preferably 3% or more, and particularly preferably 4% or more (usually, 20% or less). When the acidity is 4% or more, the vinegar can satisfy a standard of acidity of brewed vinegar defined in the Vinegar Quality Labeling Standards, and further can satisfy a standard of acidity of brewed vinegar defined in the Japanese Agricultural Standards. When the acidity of the vinegar is 4% or more, it can be, for example, 4 to 20%, further 4 to 15%, and further 4 to 10%.

The acidity (acetic acid acidity) of the vinegar is measured by a titration method in accordance with Article 4 of the Japanese Agricultural Standards. More specifically, for example, it can be measured by a method of titrating a 0.5 M sodium hydroxide standard solution to a specimen using an automatic titrator.

In addition, the brewed vinegar defined in the above-described Vinegar Quality Labeling Standards refers to brewed vinegar in Public Notice No. 1668 of the Ministry of Agriculture, Forestry and Fisheries issued on Dec. 19, 2000 finally revised in Public Notice No. 8 of the Consumer Affairs Agency issued on Aug. 31, 2011, and the brewed vinegar defined in the Japanese Agricultural Standards refers to brewed vinegar in Public Notice No. 801 of the Ministry of Agriculture, Forestry and Fisheries issued on Jun. 8, 1979 finally revised in Public Notice No. 1506 of the Ministry of Agriculture, Forestry and Fisheries issued on issued on Oct. 16, 2008 (the same shall apply hereinafter).

Furthermore, the vinegar of the present invention can be obtained by acetic acid fermentation of cereals, fruits, alcohols, vegetables, other agricultural products, or honey as a culture source (nutrient source for acetic acid bacteria) as will be described later, and can have an acidity of 4% or more as described above. When a salt-free soluble solid content is 1.2 to 4.0%, the vinegar of the present invention can satisfy a standard of brewed vinegar defined in the Vinegar Quality Labeling Standards, and further can satisfy a standard of brewed vinegar defined in the Japanese Agricultural Standards. That is, it can be labeled as brewed vinegar.

The vinegar of the present invention also includes grain vinegar, rice vinegar, rice black vinegar, apple vinegar, and grape vinegar.

The vinegar of the present invention can contain other components in addition to the water-soluble polysaccharide and acetic acid described above. As the other components, a salt content, a saccharide, an emulsifier (glycerin fatty acid ester, acetic acid monoglyceride, lactic acid monoglyceride, citric acid monoglyceride, diacetyl tartaric acid monoglyceride, succinic acid monoglyceride, polyglycerin fatty acid ester, polyglycerin condensed ricinoleic acid ester, Quillaja extract, soybean saponin, tea seed saponin, sucrose fatty acid ester, and the like), an artificial sweetener (for example, sucralose, aspartame, saccharin, and acesulfame K), a mineral (for example, calcium, potassium, sodium, iron, zinc, and magnesium, and salts thereof), a fragrance, a pH adjusting agent (for example, sodium hydroxide, potassium hydroxide, lactic acid, citric acid, tartaric acid, malic acid, and acetic acid), a cyclodextrin, an antioxidant (for example, vitamin E, vitamin C, tea extract, green coffee bean extract, chlorogenic acid, spice extract, caffeic acid, rosemary extract, vitamin C palmitate, rutin, quercetin, bayberry extract, and sesame extract), a colorant, a thickening stabilizer, and the like can be blended. These may be used singly, or two or more thereof may be used in combination.

### [2] Method for Manufacturing Vinegar

The method for manufacturing a vinegar of the present invention is characterized by including two fermentation steps S₁ and S₂. The steps are as follows, and the fermentation step S₁ is a step preceding to the fermentation step S₂. That is, the fermentation step S₂ is a step subsequent to the fermentation step S₁.

The “fermentation step S₁” is a step of culturing acetic acid bacteria selected from acetic acid bacteria belonging to the genus Acetobacter, acetic acid bacteria belonging to the genus Gluconobacter, acetic acid bacteria belonging to the genus Kozakia, acetic acid bacteria belonging to the genus Komagataeibacter, and acetic acid bacteria belonging to the genus Gluconacetobacter in a first medium to give a first culture containing a water-soluble polysaccharide selected from PS1 and PS2 PS1 and PS2 are as described above.

The acetic acid bacteria used in the fermentation step S₁ are bacteria belonging to the family Acetobacteraceae, and are acetic acid-producing bacteria. Among such acetic acid bacteria, acetic acid bacteria selected from acetic acid bacteria belonging to the genus Komagataeibacter, acetic acid bacteria belonging to the genus Kozakia, acetic acid bacteria belonging to the genus Acetobacter, acetic acid bacteria belonging to the genus Gluconobacter, and acetic acid bacteria belonging to the genus Gluconacetobacter are included, in the present invention. These may be used singly, or two or more thereof may be used in combination.

Among the above acetic acid bacteria, examples of the acetic acid bacteria of the genus Komagataeibacter include Komagataeibacter xylinus, Komagataeibacter hansenii, Komagataeibacter europaeus, and Komagataeibacter oboediens. These may be used singly, or two or more thereof may be used in combination.

Examples of the acetic acid bacteria of the genus Kozakia include Kozakia baliensis. These may be used singly, or two or more thereof may be used in combination.

Examples of the acetic acid bacteria belonging to the genus Acetobacter include Acetobacter polyoxogenes, Acetobacter tropicalis, Acetobacter indonesiensis, Acetobacter syzygii, Acetobacter cibinongensis, Acetobacter orientalis, Acetobacter pasteurianus, Acetobacter orleanensis, Acetobacter lovaniensis, and Acetobacter aceti, Acetobacter pomorum, and Acetobacter malorum. These may be used singly, or two or more thereof may be used in combination.

Examples of the acetic acid bacteria belonging to the genus Gluconobacter include Gluconobacter frateurii and Gluconobacter cerinus. These may be used singly, or two or more thereof may be used in combination.

Examples of the acetic acid bacteria belonging to the genus Gluconacetobacter include Gluconacetobacter swings ii, Gluconacetobacter xylinus, Gluconacetobacter diazotrophicus, Gluconacetobacter intermedius, Gluconacetobacter sacchari, Gluconacetobacter maltaceti, Gluconacetobacter kombuchae, and Gluconacetobacter liquefaciens. These may be used singly, or two or more thereof may be used in combination.

Among the above acetic acid bacteria, the acetic acid bacteria of the genus Komagataeibacter and the acetic acid bacteria of the genus Kozakia are preferable. More specifically, a Kozakia SP. MZ-1005 strain and a Komagataeibacter SP. MZ-077 strain are preferable. These may be used singly, or two or more thereof may be used in combination.

Of them, the Kozakia SP. MZ-1005 strain is particularly suitable for the production of PS1. In addition, the Komagataeibacter SP. MZ-077 strain is particularly suitable for the production of PS2.

The above-described Kozakia SP. MZ-1005 strain has been deposited as “NITE BP-03344” at the National Institute of Technology and Evaluation, Patent Microorganisms Depositary (No. 122, 2-5-8, Kazusakamatari, Kisarazu-shi, Chiba, Japan).

On the other hand, the Komagataeibacter SP. MZ-077 strain has been deposited as “NITE BP-03343” at the National Institute of Technology and Evaluation, Patent Microorganisms Depositary (No. 122, 2-5-8, Kazusakamatari, Kisarazu-shi, Chiba, Japan).

The acetic acid bacteria used in the fermentation step S₁ may include other acetic acid bacteria in addition to the various acetic acid bacteria described above. Examples of the other acetic acid bacteria include acetic acid bacteria belonging to the genus Acidomonas, acetic acid bacteria belonging to the genus Swaminathania, acetic acid bacteria belonging to the genus Neoasaia, acetic acid bacteria belonging to the genus Tanticharoenia, acetic acid bacteria belonging to the genus Ameyamaea, acetic acid bacteria belonging to the genus Endobacter, acetic acid bacteria belonging to the genus Granulibacter, acetic acid bacteria belonging to the genus Asaia, acetic acid bacteria belonging to the genus Acidomonas, acetic acid bacteria belonging to the genus Acidiphilium, acetic acid bacteria belonging to the genus Acidisphaera, acetic acid bacteria belonging to the genus Acidocella, acetic acid bacteria belonging to the genus Asaia, acetic acid bacteria belonging to the genus Belnapia, acetic acid bacteria belonging to the genus Craurococcus, acetic acid bacteria belonging to the genus Leahibacter, acetic acid bacteria belonging to the genus Muricoccus, acetic acid bacteria belonging to the genus Oleomonas, acetic acid bacteria belonging to the genus Paracraurococcus, acetic acid bacteria belonging to the genus Rhodopila, acetic acid bacteria belonging to the genus Roseococcus, acetic acid bacteria belonging to the genus Rubritepida, acetic acid bacteria belonging to the genus Saccharibacter, acetic acid bacteria belonging to the genus Stella, acetic acid bacteria belonging to the genus Teichococcus, and acetic acid bacteria belonging to the genus Zavarzinia. These may be used singly, or two or more thereof may be used in combination.

Origins of these acetic acid bacteria are not limited, and acetic acid bacteria used in the manufacture of a vinegar, acetic acid bacteria isolated from a manufacture site of a vinegar, acetic acid bacteria isolated from a natural environment or the like, acetic acid bacteria preserved in a culture collection institution, and the like can be appropriately used. Also, these may be used singly, or two or more thereof may be used in combination.

The first medium for culturing the acetic acid bacteria described above usually contains a carbon source. In addition, a nitrogen source can be contained. Further, a mineral source can be contained. In addition, an amino acid, a vitamin, and the like can be contained.

Among the above ones, the carbon source is a carbohydrate that is ingested by acetic acid bacteria and contributes to the production of PS1 or PS2. Examples of such a carbon source include monosaccharides such as ketose and aldose; disaccharides, trisaccharides, and higher polysaccharides to which these monosaccharides are bonded (except PS1 and PS2); sugar alcohols which are reduced products of these saccharides; starch saccharified liquids containing them; and molasses.

More specifically, examples of the ketose include ketohexoses such as fructose and sorbose, and ketopentoses such as xylulose. Examples of the aldose include aldohexoses such as glucose, mannose, and galactose, aldopentoses such as xylose and arabinose, aldotrioses such as glyceraldehyde, and aldotetroses such as erythrose. Examples of the disaccharide include sucrose, lactose, maltose, and trehalose. Examples of the trisaccharides include maltotriose. Examples of the sugar alcohol include mannitol and sorbitol. These may be used singly, or two or more thereof may be used in combination.

Among the above ones, fructose, glucose, mannose, galactose, xylose, arabinose, sucrose, maltose, trehalose, mannitol, sorbitol, glycerin, and ethanol are preferable, and fructose, glucose, mannose, galactose, arabinose, sucrose, mannitol, and sorbitol are more preferable, and at least fructose of fructose and glucose is especially preferable.

When fructose and glucose are blended in the first medium, a proportion R_{FRU} of fructose can be more than 50 mass % and 100 mass % or less when a total proportion of fructose and glucose is 100 mass %. Further, the proportion is preferably 51 mass % or more and 95 mass % or less, more preferably 52 mass % or more and 90 mass % or less, more preferably 53 mass % or more and 85 mass % or less, particularly preferably 54 mass % or more and 80 mass % or less, and especially preferably 55 mass % or more and 75 mass % or less.

The above-described carbon source may be blended as a single component, but can be blended as fruit juice or the like. For example, fruit juice of a fruit usually contains both fructose and glucose, and thus it is possible to blend both fructose and glucose into the medium by blending fruit juice of the fruit into the medium. In addition, when the proportions of fructose and glucose blended in the fruit juice are not desired blending proportions, the proportions can be adjusted by adding fructose or glucose.

Among the above ones, examples of the fruit juice include fruit juices derived from fruits such as apple, orange, grape, peach, strawberry, pear, banana, melon, kiwi, lemon, pineapple, grapefruit, cassis, acerola, blueberry, apricot, guava, plum, mango, papaya, and litchi. These may be used singly, or two or more thereof may be used in combination.

Other than the above ones, other carbon sources can be used. The other carbon sources include organic acids and alcohols. Examples of the organic acid include acetic acid, fumaric acid, citric acid, propionic acid, malic acid, malonic acid, succinic acid, lactic acid, and pyruvic acid. These may be used singly, or two or more thereof may be used in combination.

Examples of the alcohol include monohydric alcohols such as ethanol and propanol, and polyhydric alcohols such as glycerin and ethylene glycol. These may be used singly, or two or more thereof may be used in combination.

An amount of the carbon source to be blended into the medium (1 L) can be appropriately set, and can be, for example, 5 to 300 g/L, further 35 to 200 g/L, and further 75 to 150 g/L.

Among the above ones, as the nitrogen source, either an organic nitrogen source or an inorganic nitrogen source may be used. Of these, examples of the organic nitrogen source include yeast extract, dry yeast, sake lees decomposition products, sake lees, rice, bran (including various types of bran such as rice bran, red bran, medium bran, white bran, special bran, and special white bran), bran decomposition products (including decomposition products of the above-described various types of bran), wheat bran, meat extract, amino acids, protein, nucleic acids, urea, CSL, beans (soybean, peanut, etc.), processed beans, and processed bean residues (soybean meal, peanut meal, etc.). These may be used singly, or two or more thereof may be used in combination. Examples of the inorganic nitrogen source include ammonium salts (ammonium nitrate, ammonium chloride, ammonium sulfate, ammonium phosphate, etc.), nitrates (potassium nitrate, ammonium nitrate, etc.), and ammonia. These may be used singly, or two or more thereof may be used in combination.

As the nitrogen source, among the above ones, the organic nitrogen source is preferable, and, further, yeast extract, dry yeast, sake lees decomposition products, sake lees, bran, bran decomposition products, wheat bran, meat extract, and the like are preferable. When these organic nitrogen sources are blended, the amount of the organic nitrogen source to be blended into the medium (1 L) can be appropriately set, and can be, for example, 0.05 to 100 g/L, further 0.1 to 60 g/L, and further 0.2 to 40 g/L.

Examples of the mineral source include potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, zinc chloride, copper sulfate, calcium chloride, calcium carbonate, and sodium carbonate. These may be used singly, or two or more thereof may be used in combination.

A pH of the first medium is not limited, and is usually not required to be controlled in the fermentation step S₁, but can be, for example, 1 to 8, 2 to 8, or 2 to 7. When pH control is performed, hydrochloric acid, sodium hydroxide, calcium carbonate, or the like can be used.

A culture temperature is also not limited, and can be usually 15 to 45° C., and further 20 to 45° C. and further 25 to 35° C. Furthermore, a culture time is also not limited, and can be usually 3 to 15 days, and further 5 to 8 days.

The presence or absence of shaking and stirring during culture is not limited, and static culture (no shaking and no stirring) may be performed, shaking culture may be performed, or stirring culture may be performed. In addition, when the shaking culture is performed, a shaking method is not limited, and rotary shaking or reciprocating shaking may be performed. Furthermore, when the stirring culture is performed, a stirring method is not limited, and aeration type stirring or mechanical stirring may be performed. Among them, the stirring culture is preferable, and aeration stirring culture is more preferable. More specifically, deep aeration stirring culture can be used.

In a case where aeration stirring is performed, an aeration rate is not limited, and can be, for example, 0.1 to 2 vvm (aeration capacity/fermentation liquid amount/min), and can be further 0.1 to 1.5 vvm, and further 0.25 to 1.0 vvm.

In the method for manufacturing a vinegar of the present invention, an end point of the fermentation step S₁ is not limited, and the fermentation step S₁ can be terminated when fermentation proceeds to a predetermined water-soluble polysaccharide concentration. Furthermore, for example, the end point may be a time when an amount of the water-soluble polysaccharide produced in the environment is saturated.

The “fermentation step S₂” is a step of culturing acetic acid bacteria selected from acetic acid bacteria belonging to the genus Acetobacter, acetic acid bacteria belonging to the genus Gluconobacter, acetic acid bacteria belonging to the genus Kozakia, acetic acid bacteria belonging to the genus Komagataeibacter, and acetic acid bacteria belonging to the genus Gluconacetobacter in a second medium containing the first culture to give a second culture containing acetic acid and the water-soluble polysaccharide.

The acetic acid bacteria used in the fermentation step S₂ may be different from the acetic acid bacteria used in the fermentation step S₁, but can be made common therewith. That is, the case where the acetic acid bacteria are common with each other includes a case where the acetic acid bacteria used in the fermentation step S₁ are used as they are also in the fermentation step S₂. Further, there is a case where the same kind of acetic acid bacteria as the acetic acid bacteria used in the fermentation step S₁ are used also in the fermentation step S₂. Furthermore, there is a case where the fermentation step S₂ is performed by newly adding acetic acid bacteria which are not used in the fermentation step S₁ while the acetic acid bacteria used in the fermentation step S₁ are not eliminated and are used as they are also in the fermentation step S₂. the case where the acetic acid bacteria are common with each other includes a case where acetic acid bacteria not used in the fermentation step S₁ are used in the fermentation step S₂ after elimination of the acetic acid bacteria used in the fermentation step S₁.

For the acetic acid bacteria that can be used in the fermentation step S₂, the description of the acetic acid bacteria exemplified in the description of the fermentation step S₁ can be applied as it is.

The second medium in the fermentation step S₂ usually contains a carbon source and ethanol. In addition, like the first medium, the second medium can contain a nitrogen source, and further contain a mineral source, and additionally contain an amino acid, a vitamin, and the like.

For the second medium, the carbon source contained in the first medium can be used as it is. Also, the carbon source can be added as necessary. For the carbon source that can be used when the carbon source is added, the description of the carbon source exemplified in the description of the fermentation step S₁ can be applied as it is.

In addition, the second medium requires ethanol for acetic acid fermentation. When ethanol is blended into the second medium, an amount of ethanol to be blended can be appropriately set. Ethanol can be blended, for example in an amount of 1 to 15 v/v %, further in an amount of 2 to 15 v/v %, and further in an amount of 5 to 8 v/v %.

A pH of the second medium is not limited, and is usually not required to be controlled in the fermentation step S₂, but can be, for example, 1 to 8, 2 to 8, or 2 to 7.

A culture temperature is also not limited, and can be usually 15 to 45° C., and further 20 to 45° C. and further 25 to 35° C. Furthermore, a culture time is also not limited, and can be usually half a day to 14 days, and further 2 to 7 days.

The presence or absence of shaking and stirring during culture is not limited, and static culture (no shaking and no stirring) may be performed, shaking culture may be performed, or stirring culture may be performed. In addition, when the shaking culture is performed, a shaking method is not limited, and rotary shaking or reciprocating shaking may be performed. Furthermore, when the stirring culture is performed, a stirring method is not limited, and aeration type stirring or mechanical stirring may be performed. Among them, the stirring culture is preferable, and aeration stirring culture is more preferable. More specifically, deep aeration stirring culture can be used.

In a case where aeration stirring is performed, an aeration rate is not limited, and can be, for example, 0.1 to 1.5 vvm (aeration capacity/fermentation liquid amount/min), and can be further 0.1 to 1.5 vvm, and further 0.25 to 1.0 vvm.

In the method for manufacturing a vinegar of the present invention, an end point of the fermentation step S₂ is not limited, and the fermentation step S₂ can be terminated when fermentation proceeds to a predetermined acetic acid concentration. Furthermore, for example, the end point may be a time when an amount of the acetic acid produced in the environment is saturated.

The method for manufacturing a vinegar of the present invention may include only the fermentation step S₁ and the fermentation step S₂, but may include a step other than these steps. A fermentation liquid extracted from a fermentation tank can be commercialized as a vinegar through inactivation of acetic acid bacteria, aging, clarification treatment, and a sterilization step. Specifically, there are indicated a fermentation liquid extraction step of extracting a fermentation liquid from a fermentation tank, an inactivation step of inactivating acetic acid bacteria, an aging step of aging the fermentation liquid, a clarification step of clarifying the fermentation liquid, a sterilization step, and the like. These may be used singly, or two or more thereof may be used in combination.

According to the method for manufacturing a vinegar of the present invention, a vinegar containing PS1 and/or PS2 which are/is water-soluble polysaccharide(s) can be manufactured. That is, the above-described vinegar of the present invention can be manufactured. Further, a vinegar having a viscosity of 1000 mPa·s or more and an acidity of 4% or more can be manufactured.

### EXAMPLES

Hereinafter, the present invention will be described in more detail by way of examples.

### [1] Study of Fermentation Step S₁

As shown in the following Table 1, a Kozakia SP. MZ 1005 strain was inoculated, so as to attain 1 v/v %, into a sterilized first medium (pH 5.5 at the start of culture) formed by adding a carbon source in which blending proportions of fructose, glucose and sucrose were changed and 10 g/L of yeast extract, and shake-cultured under conditions of 30° C. and 150 rpm for 144 hours (6 days) to give a first culture. The viscosity of the obtained first culture was measured by the method presented in the following section “Evaluation of sample” below, and is shown in Table 1 below.

As a result, it is found that a culture having a high viscosity can be obtained by using a medium having a fructose concentration of 60 mass % or more with respect to the total sugar concentration.

TABLE 1

| | | Experimental | Experimental | Experimental |
|---|---|---|---|---|
| | | Example 1 | Example 2 | Example 3 |
| Carbon | Fructose (w/w %) | 10 | 6 | 0 |
| source | Glucose (w/w %) | 0 | 4 | 0 |
| | Sucrose (w/w %) | 0 | 0 | 10 |
| | Total concen- | 10 | 10 | 10 |
| | tration (w/w %) | | | |

| | | | |
|---|---|---|---|
| Viscosity (mPa · s) | 470 | 550 | 200 |

### [2] Analysis of Polysaccharide

First cultures of Experimental Examples 4 to 8 were obtained by the following procedures, then polysaccharides contained in each of the first cultures were separated, and each subjected to sugar composition analysis. Details of the polysaccharide separation method and the sugar composition analysis are presented in the following section “Evaluation of sample”.

### [Experimental Example 4] Non-Aeration Shaking Culture Using Fruit Juice as Nutrient Source (PS1 Production)

A Kozakia SP. MZ-1005 strain was inoculated into a sterilized medium (the medium was the same as that in Example 1, and had a pH of 5.5 at the start of culture) so as to attain 1 v/v %, and subjected to non-aeration shaking culture (flask culture) under conditions of 30° C. and 150 rpm to give a culture (first culture). No pH control was performed during this culture.

As a result, a water-soluble polysaccharide PS1 was obtained in which constituent sugars were glucose, galactose, mannose, and glucuronic acid, and a constituent ratio among the constituent sugars was 6.0:5.5:0.2:0.2.

### [Experimental Example 5] Aeration Shaking Culture Using Fruit Juice as Nutrient Source (PS1 Production)

A Kozakia SP. MZ-1005 strain was inoculated into a sterilized medium (the medium was the same as that in Example 1, and had a pH of 5.5 at the start of culture) so as to attain 1 v/v %, and subjected to aeration stirring culture (jar fermenter culture) under conditions of 30° C., 400 to 600 rpm, and 0.25 to 0.5 vvm to give a culture (first culture). No pH control was performed during this culture.

As a result, a water-soluble polysaccharide PS1 was obtained in which constituent sugars were glucose, galactose, mannose, and glucuronic acid, and a constituent ratio among the constituent sugars was 6.0:5.7:0.2:0.2.

### [Experimental Example 6] Non-Aeration Shaking Culture Using Formulated Nutrient Source (PS1 Production)

A Kozakia SP. MZ-1005 strain was inoculated into a sterilized medium (pH 5.5 at the start of culture) so as to attain 1 v/v %, and subjected to non-aeration shaking culture (flask culture) under conditions of 30° C. and 150 rpm to give a culture (first culture). No pH control was performed during this culture. The medium contains 30 g/L of fructose, 2 g/L of yeast extract, 5 g/L of trisodium citrate, 0.1 g/L of potassium dihydrogen phosphate, 0.09 g/L of dipotassium hydrogen phosphate, 0.25 g/L of magnesium sulfate, and 0.0005 g/L of ferric chloride.

As a result, a water-soluble polysaccharide PS1 was obtained in which constituent sugars were glucose, galactose, mannose, and glucuronic acid, and a constituent ratio among the constituent sugars was 6.0:5.4:0.2:0.2.

### [Experimental Example 7] Aeration Stirring Culture Using Formulated Nutrient Source (PS1 Production)

A Kozakia SP. MZ-1005 strain was inoculated into a sterilized medium (the medium was the same as that in Example 6, and had a pH of 5.5 at the start of culture) so as to attain 1 v/v %, and subjected to aeration stirring culture (jar fermenter culture) under conditions of 30° C., 400 to 600 rpm, and 0.25 to 0.5 vvm to give a culture (first culture). No pH control was performed during this culture.

As a result, a water-soluble polysaccharide PS1 was obtained in which constituent sugars were glucose, galactose, mannose, and glucuronic acid, and a constituent ratio among the constituent sugars was 6.0:5.8:0.2:0.2.

### [Experimental Example 8] Non-Aeration Shaking Culture Using Formulated Nutrient Source (PS2 Production)

A Komagataeibacter SP. MZ-077 strain was inoculated into a sterilized medium (the medium was the same as that in Example 6, and had a pH of 5.5 at the start of culture) so as to attain 1 v/v %, and subjected to non-aeration shaking culture (flask culture) under conditions of 30° C. and 150 rpm to give a culture (first culture). No pH control was performed during this culture.

As a result, a water-soluble polysaccharide PS1 was obtained in which constituent sugars were glucose, rhamnose, mannose, and glucuronic acid, and a constituent ratio among the constituent sugars was 4.0:2.1:0.2:0.2.

TABLE 2

| | | Experimental | Experimental | Experimental | Experimental | Experimental |
|---|---|---|---|---|---|---|
| | | Example 4 | Example 5 | Example 6 | Example 7 | Example 8 |
| Polysaccharide | | PS1 | PS1 | PS1 | PS1 | PS2 |
| Molar | Glucose | 6.0 | 6.0 | 6.0 | 6.0 | 4.0 |
| ratio | Galactose | 5.5 | 5.7 | 5.4 | 5.8 | — |
| | Rhamnose | — | — | — | — | 2.1 |
| | Mannose | 0.2 | 0.2 | 0.2 | 0.2 | 0.2 |
| | Glucuronic | 0.2 | 0.2 | 0.2 | 0.2 | 0.2 |
| | acid | | | | | |
| Culture | Nutrient | Apple | Apple | Formulated | Formulated | Formulated |
| condition | source | juice | juice | nutrient | nutrient | nutrient |
| | Aeration/non- | Non- | Aeration | Non- | Aeration | Non- |
| | aeration | aeration | | aeration | | aeration |
| | Stirring/ | Shaking | Stirring | Shaking | Stirring | Shaking |
| | shaking | | | | | |

### [3] Manufacture of Vinegar

Vinegars of Examples 1 to 5 were manufactured according to the following procedures.

### Example 1

(1) Fermentation Step S₁

A Kozakia SP. MZ-1005 strain was inoculated into a sterilized first medium (pH 5.5 at the start of culture) so as to attain 1 v/v %, and subjected to aeration stirring culture under conditions of 30° C., 400 to 600 rpm, and 0.25 to 0.5 vvm for 120 hours (5 days) to give a first culture. No pH control was performed during this culture. The first medium contains apple juice (Brix 10°) containing about 70 g/L of fructose and about 35 g/L of glucose, and 1 g/L of yeast extract.

As a result, a first culture containing 15.6 g/L of PS1, having a pH of 4.0 and a viscosity of 3000 mPa·s was obtained. The PS1 concentration and the viscosity were measured by the methods presented in the following section “Evaluation of sample”.

(2) Fermentation Step S₂

Ethanol and acetic acid were added to the first culture obtained in the fermentation step S₁ so as to attain 7 v/v % of ethanol and 1 w/v % of acetic acid, and the mixture was subjected to aeration stirring culture under the conditions of 30° C., 600 rpm, and 0.5 vvm for 72 hours (3 days) to give a second culture.

As a result, a vinegar containing 12.9 g/L of PS1, having an acidity of 4.9 and a viscosity of 2000 mPa·s was obtained. The acidity and the viscosity were measured by the methods presented in the following section “Evaluation of sample”.

### Example 2

(1) Fermentation Step S₁

The same culture as in Example 1 was performed to give a first culture (containing 15.6 g/L of PS1, having a pH of 4.0 and a viscosity of 3000 mPa·s).

(2) Fermentation Step S₂

Ethanol and acetic acid were added to the first culture obtained in the fermentation step S₁ so as to attain 3 v/v % of ethanol and 1 w/v % of acetic acid, and the mixture was subjected to aeration stirring culture under the conditions of 30° C., 600 rpm, and 0.5 vvm for 24 hours (1 days) to give a second culture.

As a result, a vinegar containing 14.0 g/L of PS1, having an acidity of 3.3 and a viscosity of 2400 mPa·s was obtained. The acidity and the viscosity were measured by the methods presented in the following section “Evaluation of sample”.

### Example 3

(1) Fermentation Step S₁

A Kozakia SP. MZ-1005 strain was inoculated into a sterilized first medium (pH 5.5 at the start of culture) so as to attain 1 v/v %, and subjected to aeration stirring culture under conditions of 30° C., 400 to 600 rpm, and 0.25 to 0.5 vvm for 148 hours (6 days) to give a first culture. No pH control was performed during this culture. The first medium contains apple juice (Brix 10°) containing about 70 g/L of fructose and about 35 g/L of glucose, and 1 g/L of yeast extract.

As a result, a first culture containing 15.7 g/L of PS1, having a pH of 3.8 and a viscosity of 2900 mPa·s was obtained. The PS1 concentration and the viscosity were measured by the methods presented in the following section “Evaluation of sample”.

(2) Fermentation Step S₂

Ethanol was added to the first culture obtained in the fermentation step S₁ so as to attain 4 v/v % of ethanol, and the mixture was subjected to aeration stirring culture under the conditions of 30° C., 600 rpm, and 0.5 vvm for 24 hours (1 days) to give a second culture.

As a result, a vinegar containing 14.0 g/L of PS1, having an acidity of 1.6 and a viscosity of 2200 mPa·s was obtained. The acidity and the viscosity were measured by the methods presented in the following section “Evaluation of sample”.

### Example 4

(1) Fermentation Step S₁

A Komagataeibacter SP. MZ-077 strain was inoculated into a sterilized first medium (pH 6.0 at the start of culture) so as to attain 1 v/v %, and subjected to aeration stirring culture under conditions of 30° C., 400 to 600 rpm, and 0.25 to 0.5 vvm for 120 hours (5 days) to give a first culture. No pH control was performed during this culture. The first medium contains apple juice (Brix 10°) containing about 70 g/L of fructose and about 35 g/L of glucose, 1 g/L of yeast extract, and 2 g/L of a red bran decomposition product.

As a result, a first culture containing 13.8 g/L of PS2, having a pH of 5.0 and a viscosity of 4000 mPa·s was obtained. The PS2 concentration and the viscosity were measured by the methods presented in the following section “Evaluation of sample”.

(2) Fermentation Step S₂

Ethanol and acetic acid were added to the first culture obtained in the fermentation step S₁ so as to attain 8 v/v % of ethanol and 1 w/v % of acetic acid, and the mixture was subjected to aeration stirring culture under the conditions of 30° C., 600 rpm, and 0.5 vvm for 168 hours (7 days) to give a second culture.

As a result, a vinegar containing 13.4 g/L of PS2, having an acidity of 4.8 and a viscosity of 3100 mPa·s was obtained. The acidity and the viscosity were measured by the methods presented in the following section “Evaluation of sample”.

### Example 5

(1) Fermentation Step S₁

The same culture as in Example 4 was performed to give a first culture (containing 13.8 g/L of PS2, having a pH of 5.0 and a viscosity of 4000 mPa·s).

(2) Fermentation Step S₂

Ethanol and acetic acid were added to the first culture obtained in the fermentation step S₁ so as to attain 4 v/v % of ethanol and 1 w/v % of acetic acid, and the mixture was subjected to aeration stirring culture under the conditions of 30° C., 600 rpm, and 0.5 vvm for 72 hours (3 days) to give a second culture.

As a result, a vinegar containing 13.5 g/L of PS2, having an acidity of 3.1 and a viscosity of 3300 mPa·s was obtained. The acidity and the viscosity were measured by the methods presented in the following section “Evaluation of sample”.

### [4] Evaluation of Sample (Vinegar/First Culture)

(1) Measurement of Acidity (in Terms of Acetic Acid)

The acidity was measured by the following procedures.

An automatic titrator COM-1600 (Hiranuma Sangyo Co., Ltd.) was used to neutralize and titrate 5 ml of each vinegar with 0.5 M sodium hydroxide until the pH reached 8.2, and then a titer was converted into acetic acid acidity % (w/v) by the following formula. Furthermore, a value converted into mass % was defined as acidity (in terms of acetic acid) in consideration of a specific gravity of the vinegar for the calculated acetic acid acidity % (w/v).

Acetic acid acidity % (w/v)=0.03×(T−B)×F/V×100
T: titer (ml) of 0.5 mol/L sodium hydroxide standard solution in each vinegar
B: titer (ml) of 0.5 mol/L sodium hydroxide standard solution in blank test
F: factor of 0.5 mol/L sodium hydroxide standard solution
V: amount (ml) of collected vinegar 0.03: weight (g) of acetic acid corresponding to 1 ml of 0.5 mol/L sodium hydroxide solution

(2) Measurement of pH

The pH of a sample was measured by a glass electrode method.

A pH meter F-51 (manufactured by HORIBA, Ltd.) was used for the measurement.

(3) Measurement of Viscosity

The viscosity was measured by the following procedures.

A BII-type viscometer (BMII (manufactured by Toki Sangyo Co., Ltd.)) equipped with a No. 3 rotor was used to measure the viscosity of the sample (25° C.) at a rotor rotation number of 30 rpm.

(4) Measurement of PS1 Concentration and PS2 Concentration

A supernatant was collected from the sample by centrifugation (12000 G, 10 min, 4° C.). To 0.5 mL of the supernatant, added was 1.5 mL of 99.5% cold ethanol, and a precipitate was collected by centrifugation (12000 G, 10 min, 4° C.). The collected precipitate was washed through addition of 1 mL of 75% cold ethanol, and then collected by centrifugation (12000 G, 10 min, 4° C.). The obtained precipitate was dissolved in 0.5 mL of ultrapure water (so-called MillQ water), and the PS1 or PS2 concentration was measured using a phenol sulfuric acid method.

(5) Separation of Polysaccharide

Each of the first cultures obtained in Experimental Examples 4 to 8 was diluted with ultrapure water, followed by centrifugation and diatomaceous earth filtration to remove bacterial cells. Thereafter, a 70% ethanol aqueous solution was added to the filtrate to give a precipitate. The obtained precipitate was collected and redissolved with water. Then, a 5% CTAB aqueous solution (aqueous hexadecyltrimethylammonium bromide solution) was added (added until no precipitate was generated) to give a precipitate, and then the precipitate was collected by centrifugation. Thereafter, the collected precipitate was redissolved with a 20% NaCl aqueous solution. Next, dialysis was performed with ultrapure water, and then a 70% ethanol aqueous solution was added to give a precipitate. The obtained precipitate was collected by centrifugation and then resuspended in water. Thereafter, dialysis was performed again with ultrapure water, and then freeze-drying was performed to give polysaccharide samples produced in Experimental Examples 4 to 8.

(6) Sugar composition analysis of polysaccharide

Each of the polysaccharide samples of Experimental Examples 4 to 8 obtained in the above item (5) was solubilized with ultrapure water so as to attain 2 mg/ml, subjected to acid hydrolysis and fluorescent labeling, and then to ultra-high performance high separation liquid chromatography (UPLC). Analysis conditions are as follows. For quantitative analysis of constituent sugars, 13 kinds of sugar standard products which will be described below were used.
Apparatus: model “ACQUITY UPLC H-Class”, manufactured by Waters Corporation
Column: model “ACQUITY UPLC BEH C18, 2.1 mm×100 mm” manufactured by Waters Corporation
Mobile phase: A/0.2 M potassium borate buffer (pH 8.9), B/acetonitrile
Flow rate: 0.7 mL/min
Column temperature: 50° C.
Detection: FLR 305 nm, 360 nm
Injection vol: 1 μL
Time program: B conc. 4.5% (0 min)−4.5% (8 min)−9.0% (15 min)
Sugar standard products: glucuronic acid (GlcA), galacturonic acid (GalA), galactose (Gal), mannose (Man), glucose (Glc), arabinose (Ara), ribose (Rib), xylose (Xyl), N-acetylmannosamine (ManNAc), N-acetylglucosamine (GlcNAc), rhamnose (Rha), fucose (Fuc), N-acetylgalactosamine (GalNAc)

(7) NMR Analysis of Polysaccharide

The polysaccharide PS1 produced under the same conditions as in the Experimental Examples was separated in the same manner as in the above item (5). To the PS1 (3.57 mg), added was 1000 ml of D20, and the mixture was warmed to 60° C. to give a PS1 solution. As an NMR sample, 600 μL of the liquid was fractionated, and subjected to structure analysis by NMR under the following analysis conditions. As a result, it was found that PS1 includes a structure “Glcβ1→6Glcβ1→6Gal→”.
Apparatus: 500 MHz NMR, manufactured by Agilent Technologies, model: “Unity INOVA 500”
Measurement temperature: 60° C.
Other conditions: Water elimination pulse irradiation
Fourier transformation and analysis: applied to the obtained NMR FID data using ACD/NMR Processor (http://www.acdlabs.com) Measured species and number of times of integration
   1H NMR/number of times of integration: 8
   COSY/number of times of integration: 1
   HSQC, number of times of integration: 32
   TOCSY, number of times of integration: 32
   HSQC-TOCSY, number of times of integration: 64
   HMBC, number of times of integration: 64

The measured species are as follows. COSY (Correlation Spectroscopy), HSQC (Hetero Nuclear Single Quantum Coherence), TOCSY (Totally Correlated Spectroscopy), HSQC-TOCSY, HMBC (Heteronuclear Multiple Bond Coherence)

In the present specification, the description “XX to YY” means “XX or more and YY or less”.

Further, the present invention is not limited to the above specific examples. These examples are merely illustrated for convenience of description, and the present invention is not limited to these examples in any sense, and can be variously modified within the scope of the present invention according to the purpose and application. All publications, patents, and patent applications cited herein are incorporated herein by reference in their entirety.

The vinegar of the present invention is widely used in the field of foods. More specifically, the vinegar of the present invention can be suitably used as a viscous liquid seasoning because the vinegar itself has a high viscosity. In this case, it can exhibit excellent adhesiveness to foods. In addition, the vinegar of the present invention itself has a high viscosity, and thus can be used as a raw material for a viscous liquid seasoning (sauce, ketchup, dipping sauce, dressing sauce, or the like) having a sour taste. In this case, it can be used as a food raw material capable of imparting a sour taste and a vinegar flavor while suppressing a decrease in viscosity.

What is claimed is:

1. A method for manufacturing an acetic acid bacterium culture, comprising a fermentation step S1 below:
the fermentation step S1: a step of culturing acetic acid bacteria belonging to a genus Kozakia to give a first culture comprising PS1 below, or culturing acetic acid bacteria belonging to a genus Komagataeibacter to give a first culture comprising PS2 below, or culturing acetic acid bacteria belonging to the genus Kozakia and acetic acid bacteria belonging to the genus Komagataeibacter to give a first culture comprising PS1 and PS2 below,
in a first medium comprising at least fructose of fructose and glucose, in which a proportion R_{FRU} of the fructose is more than 50 mass % and 100 mass % or less when a total proportion of the fructose and the glucose is 100 mass %,
PS1: a water-soluble polysaccharide in which constituent sugars are glucose, galactose, mannose, and glucuronic acid, and a constituent ratio among the constituent sugars is 6.0:5.0 to 6.5:0.05 to 0.4:0.05 to 0.4,
PS2: a water-soluble polysaccharide in which constituent sugars are glucose, rhamnose, mannose, and glucuronic acid, and a constituent ratio among the constituent sugars is 4.0:1.5 to 2.5:0.05 to 0.4:0.05 to 0.4.

2. The method for manufacturing the acetic acid bacterium culture according to claim 1, wherein the culture in the fermentation step S1 is either stirring culture under aeration conditions or static culture under non-aeration conditions.

3. The method for manufacturing the acetic acid bacterium culture according to claim 1, wherein the first culture has a pH of 3.8 or more.

4. The method for manufacturing the acetic acid bacterium culture according to claim 1, wherein, in the fermentation step S1, the acetic acid bacteria belonging to the genus Kozakia are a Kozakia SP. MZ-1005 strain, and the acetic acid bacteria belonging to the genus Komagataeibacter are a Komagataeibacter SP. MZ-077 strain.

5. The method for manufacturing the acetic acid bacterium culture according to claim 1, further comprising a fermentation step S2 below after the fermentation step S1:
the fermentation step S2: a step of culturing acetic acid bacteria selected from acetic acid bacteria belonging to a genus Acetobacter, acetic acid bacteria belonging to a genus Gluconobacter, acetic acid bacteria belonging to the genus Kozakia, acetic acid bacteria belonging to the genus Komagataeibacter, and acetic acid bacteria belonging to a genus Gluconacetobacter in a second medium comprising the first culture to give a second culture comprising acetic acid and the PS1 and/or the PS2.

6. The method for manufacturing the acetic acid bacterium culture according to claim 5, wherein the acetic acid bacteria used in the fermentation step S1 and the acetic acid bacteria used in the fermentation step S2 are common with each other.

7. The method for manufacturing the acetic acid bacterium culture according to claim 5, wherein the acetic acid bacteria used in the fermentation step S1 are continuously used in the fermentation step S2.

8. The method for manufacturing the acetic acid bacterium culture according to claim 5, further comprising a step of adding ethanol to the second medium to manufacture an acetic acid bacterium culture having an acidity of 0.5% or more.

9. The method for manufacturing the acetic acid bacterium culture according to claim 5, wherein the second culture has a viscosity of 1000 mPa·s or more and an acidity of 4% or more.

10. The method for manufacturing the acetic acid bacterium culture according to claim 5, wherein a medium not added with ethanol is used as the first medium in the fermentation step S1, a medium added with ethanol is used as the second medium in the fermentation step S2, and the fermentation step S1 and the fermentation step S2 are carried out in series, so that the acetic acid bacteria used in the fermentation step S1 are continuously used in the fermentation step S2 to produce the polysaccharide through the culture of the acetic acid bacteria and to produce the acetic acid through the culture of the acetic acid bacteria.

11. The method for manufacturing the acetic acid bacterium culture according to claim 5, wherein the culture of the acetic acid bacteria in the fermentation step S2 is either stirring culture under aeration conditions or static culture under non-aeration conditions.

12. The method for manufacturing the acetic acid bacterium culture according to claim 5, further comprising, after the fermentation step S2, at least one steps selected from a step of inactivating the acetic acid bacteria, a step of aging a fermentation liquid, a step of clarifying a fermentation liquid, and a step of sterilizing a fermentation liquid.

13. The method for manufacturing an acetic acid bacterium culture according to claim 5,
wherein the fermentation step S1 is a step of performing fermentation through stirring culture under aeration conditions or static culture under non-aeration conditions until a viscosity of the first culture reaches 1000 mPa·s or more, using a medium not added with ethanol as the first medium, and
wherein the fermentation step S2 comprises a stage of adding ethanol to the second medium, and is a step of performing stirring culture under aeration conditions or static culture under non-aeration conditions of acetic acid bacteria common with the acetic acid bacteria used in the fermentation step S1 to give a second culture containing the PS1 and/or the PS2 and having an acidity of 4% or more.

14. The manufacture method according to claim 13, wherein the fermentation step S1 and the fermentation step S2 are carried out by deep aeration stirring culture.

15. A method for increasing a viscosity of an acetic acid bacterium culture by incorporating a fermentation step S1 below:
a fermentation step S1: a step of culturing acetic acid bacteria belonging to a genus Kozakia to give PS1 below, or culturing acetic acid bacteria belonging to a genus Komagataeibacter to give a first culture comprising PS2 below, or culturing acetic acid bacteria belonging to the genus Kozakia and acetic acid bacteria belonging to the genus Komagataeibacter to give a first culture comprising PS1 and PS2 below,
in a first medium comprising at least fructose of fructose and glucose, in which a proportion R_{FRU} of the fructose is more than 50 mass % and 100 mass % or less when a total proportion of the fructose and the glucose is 100 mass %,
PS1: a water-soluble polysaccharide in which constituent sugars are glucose, galactose, mannose, and glucuronic acid, and a constituent ratio among the constituent sugars is 6.0:5.0 to 6.5:0.05 to 0.4:0.05 to 0.4,
PS2: a water-soluble polysaccharide in which constituent sugars are glucose, rhamnose, mannose, and glucuronic acid, and a constituent ratio among the constituent sugars is 4.0:1.5 to 2.5:0.05 to 0.4:0.05 to 0.4.

16. A method for increasing both a viscosity and an acidity of an acetic acid bacterium culture, as a result of increasing a viscosity of an acetic acid bacterium culture by the method according to claim 15, and then increasing an acidity of the acetic acid bacterium culture by incorporating a fermentation step S2 below:
the fermentation step S2: a step of blending ethanol in a second medium comprising the first culture, and culturing acetic acid bacteria selected from acetic acid bacteria belonging to a genus Acetobacter, acetic acid bacteria belonging to a genus Gluconobacter, acetic acid bacteria belonging to the genus Kozakia, acetic acid bacteria belonging to the genus Komagataeibacter, and acetic acid bacteria belonging to a genus Gluconacetobacter in the second medium to give a second culture comprising acetic acid and the PS1 and/or the PS2.

17. A method for providing an acetic acid bacterium culture having a viscosity increased, by the method according to claim 16, so as to reach 1000 mPa·s or more due to the PS1 and/or the PS2 contained therein, although the acetic acid bacterium culture corresponds to brewed vinegar as defined in Vinegar Quality Labeling Standards (Public Notice No. 1668 of the Ministry of Agriculture, Forestry and Fisheries issued on Dec. 19, 2000, finally revised in Public Notice No. 8 of the Consumer Affairs Agency issued on Aug. 31, 2011).

18. An acetic acid bacterium culture comprising PS1 below and/or PS2 below:
the PS1: a water-soluble polysaccharide in which constituent sugars are glucose, galactose, mannose, and glucuronic acid, and a constituent ratio among the constituent sugars is 6.0:5.0 to 6.5:0.05 to 0.4:0.05 to 0.4
the PS2: a water-soluble polysaccharide in which constituent sugars are glucose, rhamnose, mannose, and glucuronic acid, and a constituent ratio among the constituent sugars is 4.0:1.5 to 2.5:0.05 to 0.4:0.05 to 0.4.

19. The acetic acid bacterium culture according to claim 16, which has an acidity of 0.5% or more.

20. The acetic acid bacterium culture according to claim 16, which has a viscosity of 1000 mPa·s or more and an acidity of 4% or more.
